(19) United States
(12) Patent Application Publication
(10) Pub. No.: US 2016/0115432 A1
(43) Pub. Date: Apr. 28, 2016

(54) Symbiotic Algae System

(71) Applicant: Anju Dahiya, Burlington, VT (US)

(72) Inventor: Anju Dahiya, Burlington, VT (US)

(73) Assignee: GSR SOLUTIONS, LLC, Burlington, VT (US)

(21) Appl. No.: 14/932,218

(22) Filed: Nov. 4, 2015

Related U.S. Application Data
(63) Continuation-in-part of application No. 14/888,986.
(60) Provisional application No. 62/079,135, filed on Nov. 13, 2014.
(60) Provisional application No. 62/067,042, filed on Oct. 22, 2014.
(60) Provisional application No. 62/067,049, filed on Oct. 22, 2014.

(51) Int. Cl. C12M 1/00; C12M 1/26; C12M 1/12

(52) U.S. Cl. CPC C12M 21/02; C12M 29/00; C12M 47/10; C12M 25/00; C12M 33/00

(57) ABSTRACT

According to present disclosure, there is disclosed an algae growth and cultivation system that provides a cost-efficient means of producing algae biomass as feedstock for algae-based products, such as, biofuel manufacture, and desirably impacts alternative/renewable energy production, nutrient recovery from waste streams, and valued byproducts production. The system as discussed herein is an integrated systems approach to wastewater treatment, algal strains selection for byproducts production, and recycle of algal-oil extraction waste or additional algae harvested as feedstock for fertilizer production. Embodiments of a system as discussed herein present an economically viable algae production system and process that allows algae-derived products such as biofuels, fertilizer, etc. to compete with petroleum products in the marketplace.

### RELATED APPLICATION DATA

This application is a continuation-in-part application of U.S. patent application Ser. No. 14/888,986, filed Nov. 4, 2015, and entitled “Symbiotic Algae System with Looped Reactor”, which is a national stage entry of PCT Application No. PCT/US15/56344, filed on Oct. 20, 2015, and entitled “Symbiotic Algae System with Looped Reactor”, which claims priority to U.S. provisional application Ser. No. 62/067,049, filed Oct. 22, 2014, and entitled “Symbiotic Algae System with Looped Reactor”, U.S. provisional application Ser. No. 62/067,042, filed Oct. 22, 2014, and entitled “Symbiotic Algae System”, and U.S. provisional application Ser. No. 62/079,135, filed Nov. 13, 2014, and entitled “Algal Growth System Process Utilizing Intermediate Products of Consolidated Bioprocessing Process or Anaerobic Digestion Process”, each of the aforementioned applications are hereby incorporated by reference herein in their entirety.

### FIELD OF THE INVENTION

The present invention relates to algae growth systems and in particular to a Symbiotic Algae System.

### BACKGROUND

Mass cultivation of algae has been used for creating nutritional supplements, fertilizer, and food additives. Commercial growth of algae has also been explored to create biologically-derived energy products such as biodiesel, bioethanol, and hydrogen gas. As a biofuel feedstock, algae provide multiple environmental benefits and present significant advantages over traditional plants/crops used for biofuel production (e.g., corn, sugarcane, switch-grass, etc.). For example, unlike traditional food crops that are being used to produce biofuels (e.g., corn, sugarcane, etc.), algae does not compete with food and water resources; it grows significantly faster than traditional crops used for biodiesel; algae produce up to 300 times more oil than traditional crops on an area basis; algae fuel has properties (low temperature and high energy density) of that make it suitable as jet fuel; and algae can be produced so as to provide a nearly continuous supply of fuel. Moreover, algae can treat industrial, municipal and agricultural wastewaters, capture carbon-dioxide, and provide valuable byproducts, such as, but not limited to, protein-rich feed for farm animals, organic fertilizer, and feedstock for producing biogas.

Algal biomass can accumulate up to 50% carbon by dry weight, therefore producing 100 tons of algal biomass fixes roughly 183 tons of CO₂—providing a tremendous potential to capture CO₂ emissions from power plant flue gases and other fixed sources. Ideally, biodiesel from algae can be carbon neutral, because all the power needed for producing and processing the algae could potentially come from algal biodiesel and from methane produced by anaerobic digestion of the biomass residue left behind after the oil has been extracted.

The successful role of algae in wastewater treatment has been documented since the early 1950s, and algal wastewater treatment systems are known to utilize the extra nutrients including nitrogen, phosphorus, potassium, heavy metals and other organic compounds from wastewater. For example, an algal turf scrubber system feeding algae a diet of dairy manure can recover over 95% of the nitrogen and phosphorous in the manure wastewater. Additionally, lipid/oil productivity occurs in algal wastewater treatment systems, but there are few, if any, known robust algae strain(s) for oil production that use wastewater as a primary feedstock. For example, a polyculture (dominated by Rhizoclonium sp.) used in algal turf systems for treating dairy and swine wastewater had very low lipids/oil content (fatty acids contents of 0.6% to 1.5% of dry algae weight) and other researchers have reported 2.8 g/m² per day of lipid productivity from algal poly culture combined with dairy wastewater treatment.

Algae's other byproducts can also be beneficial. For example, the value of algae as food was explored as early as 1950s, and some have demonstrated the concept by raising baby chickens to adults on twenty percent (20%) algae fortified feed (grown on pasteurized chicken manure). The antibiotic Chlorellin extracted from Chlorella during World War II marked the start of algae based pharmaceutical and nutraceutical industry that led to the Japanese Chlorella production facilities during 1960s, further leading to current production of Chlorella, Spirulina, Dunaliella and Hematococus at commercial scales. Fertilizers from algae have also shown equivalence to commercial organic fertilizers in terms of plant mass and nutrient content.

Despite all of the aforementioned benefits, algae biomass production and the production of algal oil (i.e., biofuels from algae) are primarily hampered by the high cost of producing algae biomass (currently either requiring large amounts of land/water and/or large sterile facilities). There have been attempts to offset this high cost by using the various traits of algae to their greatest benefit. For example, biofuel production from algae has been combined with waste-water treatment (as discussed above) and has been shown to be 40% more cost effective than the best conventional alternatives, but still has not been economically viable due to low lipid production. As another example, entities have attempted to vary the type of cultures used—for example, algae monoculture (requiring sterile conditions) versus polyculture-based wastewater treatment. However, the results of these trials have not proven themselves. Other disadvantages of current algae biomass production include, but are not limited to, the availability of low cost throughput sugar feed-stocks for growing algae, treating effluent created during production, and the requirement of nitrogen and phosphorus supplements. Until such time as these algae production related issues are solved, production of oil feedstock from algae is likely to remain commercially infeasible.

For this reason, the system and process disclosed herein addresses the challenges involved in materializing the cost-efficient algae-based on a robust, easily adaptable, environmentally friendly system that is capable of growing algae biomass at commercial scales for biofuel, fertilizer, animal feed, and other byproducts. The symbiotic algae system and process disclosed herein also holds great potential for industries, farms and municipalities, especially dairy farms and breweries, because the systems allows these entities to more efficiently and effectively meet government standards for handling and recycling of wastes.

### SUMMARY

In an exemplary aspect, a symbiotic algae system comprises a first algal growth component, wherein the first algal growth component includes a heterotrophic organism, and wherein the first algal growth component produces a first effluent and an off-gas; and a second algal growth component is fluidly coupled to the first algal growth component, and the second algal growth component including at least one organism from the group of: a photoautotrophic organism, a mixotrophic organism, and a heterotrophic organism, and wherein the second algal growth component receives, as an input, the first effluent and the off-gas and produces a second effluent.

In another exemplary aspect, a symbiotic algae system comprises a first algal growth component, wherein the first algal growth component includes a heterotrophic organism, and wherein the first algal growth component produces an first effluent and an off-gas; and a second algal growth component fluidly coupled to the first algal growth component, wherein the second algal growth component includes at least one organism from the group of: a photoautotrophic organism, a mixotrophic organism, and a heterotrophic organism, and wherein the second algal growth component receives, as a first input, the first effluent and the first off-gas and produces an second effluent and a second off-gas; and wherein the second effluent and the second off-gas are received as inputs to the first algal growth component.

In yet another exemplary aspect, a symbiotic algae system comprises a waste nutrient preparation sub-system; an algal culturing system including: a first algal growth component fluidly coupled to the waste-nutrient preparation sub-system, wherein the first algal growth component includes a heterotrophic organism, and wherein the first algal growth component produces a first effluent and an off-gas; and a second algal growth component, wherein the second algal growth component includes at least one organism from the group of: a photoautotrophic organism, a mixotrophic organism, and a heterotrophic organism, and wherein the second algal growth component receives, as an input, the effluent and the off-gas and produces a second effluent; and an algal harvesting system fluidly coupled to the algal culturing system; an algal biomass processing system fluidly coupled to the algal harvesting system; and a byproducts system fluidly coupled to the algal biomass processing system.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary symbiotic algae system according to an embodiment of the present invention;

FIG. 2 is a block diagram of an algal core suitable for use with an exemplary symbiotic algae system such as the systems shown in FIGS. 1 and 5;

FIG. 3 is a block diagram of another algal core suitable for use with an exemplary symbiotic algae system such as the systems shown in FIGS. 1 and 5;

FIG. 4 is a block diagram of another algal core suitable for use with an exemplary symbiotic algae system such as the systems shown in FIGS. 1 and 5;

FIG. 5 is a block diagram of a portion of an exemplary symbiotic algae system according to another embodiment of the present invention;

FIG. 6 is a chart of algal cell density showing the optical density over time for a test core according to an embodiment of the present invention and a control;

FIG. 7 is a block diagram of a portion of an exemplary symbiotic algae system suitable for removing contaminants according to another embodiment of the present invention

FIG. 8 is a block diagram of an exemplary process of removing contaminants from a waste stream according to an embodiment of the present invention; and

FIG. 9 is a table showing prior art energy returns for biodiesel using various feed-stocks.

### DETAILED DESCRIPTION

A symbiotic algae system according to present disclosure provides a cost-efficient means of producing algae biomass for many applications, such as, but not limited to, as feedstock for biofuel manufacture and desirably impacts alternative/renewable energy production, nutrient recovery from waste streams, and valued byproducts production (nutraceuticals, pharmaceuticals, animal feed etc.). A symbiotic algae system as discussed herein is an integrated systems approach to wastewater treatment, algal strains selection for oil production, CO₂ capture or nutrient capture from heterotrophic processes, and recycle of algal-oil extraction waste as feedstock for biogas production. Embodiments of a symbiotic algae system as discussed herein present an economically viable algae production system and process that allows algae-derived biofuels to compete with petroleum products in the marketplace.

A symbiotic algae system as discussed herein is, at a high level, a scalable process for cultivating algae biomass, in which a heterotrophic (i.e., non-light dependent) algal growth strain is used to provide carbon dioxide and/or effluent to a photoautotrophic or mixotrophic or a combination of the three cultivation processes (i.e., photoautotrophic, mixotrophic, and heterotrophic) while concomitantly producing algae biomass or lipids for biofuel production. In certain embodiments, the photoautotrophic or mixotrophic or heterotrophic cultivation portion of the symbiotic algae system may result in the cultivation of additional algae biomass, but could include (alternatively or additionally) the cultivation of any photoautotrophically or mixotrophically grown microbial plant matter that requires carbon dioxide and/or effluent containing nutrients, such as nitrogen, phoshporus and organic carbon. As will be discussed in more detail below, the symbiotic algae system can efficiently use nutrients from both commercial and/or other waste streams for the production of lipids for use with biofuels, and as such, the energy return on investment scenarios are significantly higher than previously considered possible. This symbiotic algae system provides a robust scalable option which has improved cost efficiencies due to production of additional desirable byproducts such as fertilizer.

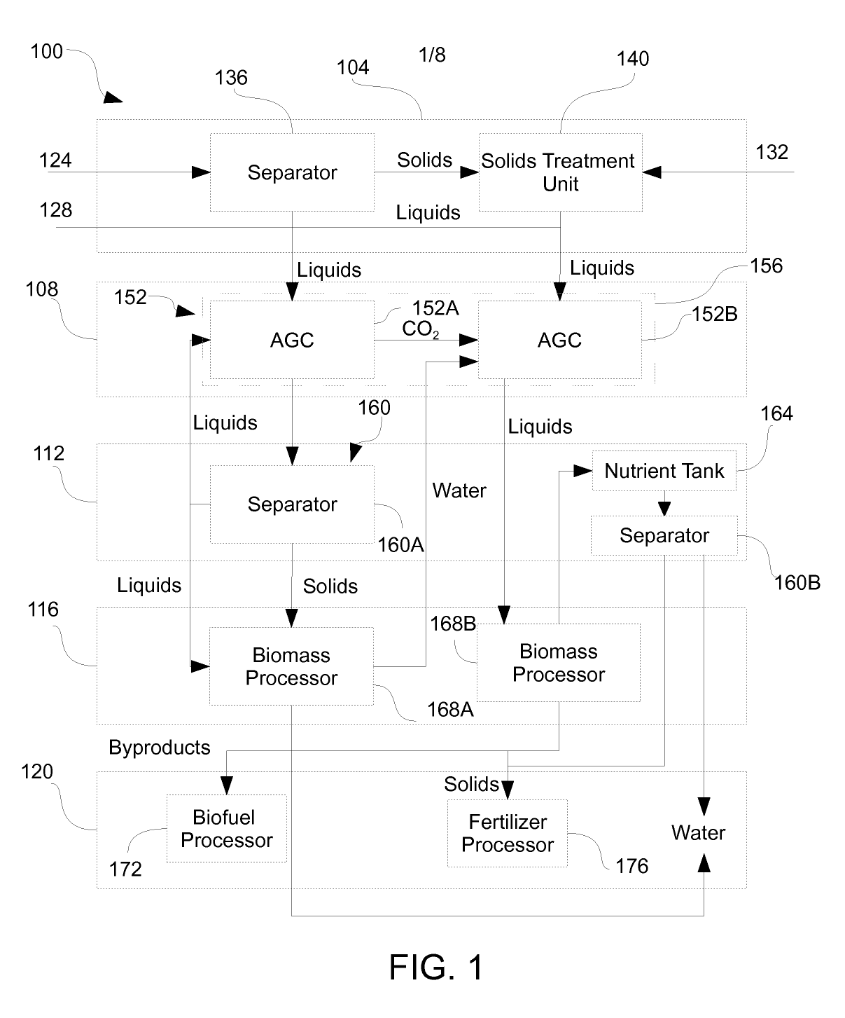

Turning now to the figures, and specifically with reference to FIG. 1, there is shown a symbiotic algae system (SAS) 100. In an exemplary embodiment, SAS 100 includes, at a high level, a waste nutrient preparation sub-system 104, an algal culturing system 108, an algal harvesting system 112, an algal biomass processing system 116, and a byproducts system 120.

Waste nutrient preparation sub-system 104 is generally configured to treat incoming feedstocks (e.g., manure, municipal waste) for the rest of SAS 100. The design and configuration of waste nutrient preparation sub-system 104 depends on the desired inputs for SAS 100. As shown in FIG. 1, waste nutrient sub-system 104 includes three inputs: an effluent input 124, a water input 128, and a waste input 132. Effluent input 124 can generally be any nutrient rich liquid waste before or after single or multiple pre-treatments, for example, dairy farm effluent, agricultural wastewater streams, brewery liquid waste streams, municipal waste, food waste, etc. Effluent input 124 is fed into a separator 136 that separates the effluent solids and liquids, using methods such as settling, filtration, or via centrifugal separators. The solids can then be fed to a solids treatment unit 140, such as a digester, which can, among other things, break down the solids into a feed stream suitable for further use within SAS 100, such as a source of carbon dioxide and sugars, or into other byproducts (e.g., biogas, fertilizers, etc.). Solids treatment unit 140 can also accept waste input 132 for processing solids treated by unit 140. The output of solids treatment unit 140 and the liquid effluent separated by separator 136 may be combined with fresh water input 128 to prepare the feedstock for algae culturing system 108 that includes one or more algae growth components (AGC) 152, e.g., AGC 152A and AGC 152B.

In an exemplary embodiment, waste nutrient preparation sub-system 104 is a manure settling and solid's preparation unit that outputs liquid manure waste to algal culturing system 108. In this embodiment, manure is combined with water run-off (e.g., fresh water input 128) and collected in a large separation tank (e.g., separator 136). The denser solids are allowed to sink to the bottom (or in certain embodiments are mechanically separated) and the output liquid manure water is pumped from the tank. In an exemplary embodiment, solid wastes, for example, ligno-cellulosic material such as grain spoilage or grasses, is pretreated in solids treatment unit 140 with or without manure effluent to prepare the nutrients (e.g., different forms of nitrogen or phoshporus or sugars or organic carbon) for algal culturing in algae culturing system 108.

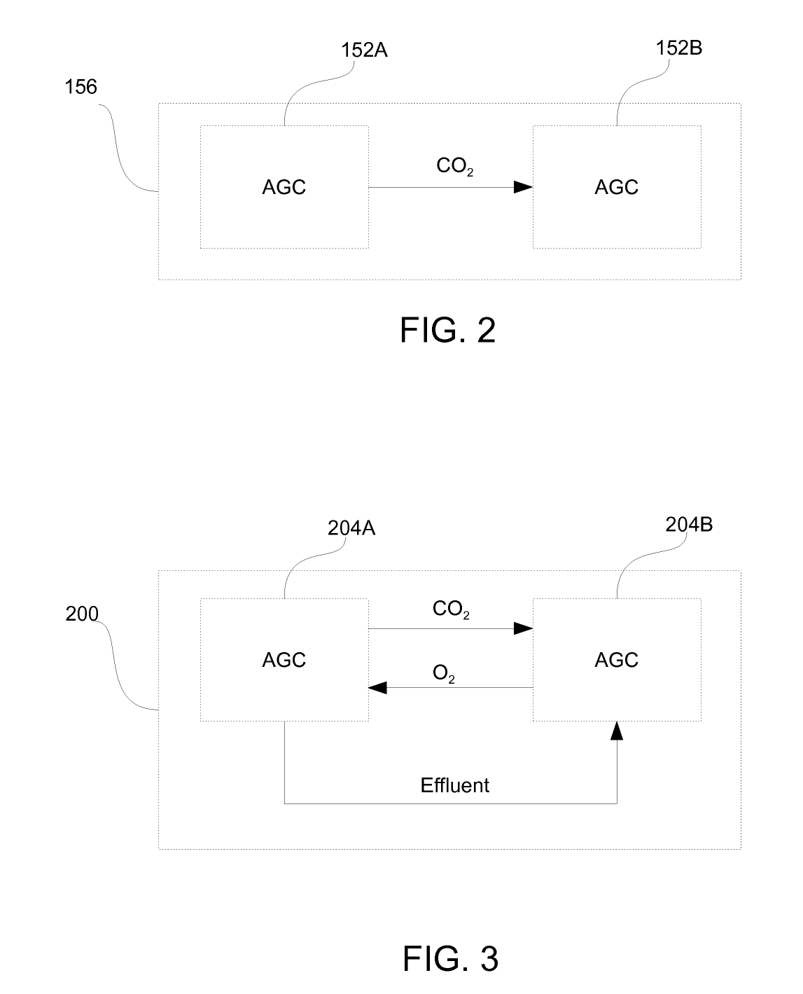

Algal culturing system 108 is generally configured to grow algal biomass from numerous nutrient and/or waste streams. In an exemplary embodiment, algal culturing system includes an algal core 156 (FIG. 2), which can include an AGC 152A that is coupled to, and mutually supports, an AGC 152B. In an exemplary embodiment, AGC 152A is an organic carbon source fed heterotrophic algae and AGC 152B is one or more of a photoautotrophic, mixotrophic, and heterotrophic algae. In general, heterotrophic algal production produces higher amounts of oil/lipids compared to its lighted dependent counterpart (e.g., mixotrophic, photoautotrophic), however it is limited in its ability to capture nutrients or other desired extracts and also generates effluent that typically requires treatment. Algal cultivating system 108 combines the two complementary approaches thereby providing a system that can produce high amounts of oil/lipids and can capture nutrients for byproducts such as fertilizer production that can offset the costs of algal biomass production. For example, the algae Chlorella vulgaris can remove up to about 20.8% of phosphate under autotrophic conditions, up to about 17.8% under heterotrophic conditions, and up to about 20.9% under mixotrophic conditions after 5 days when grown in synthetic wastewater. Algal culturing system 108, in certain embodiments described herein, can capture the remaining nutrients left after the heterotrophic algal growth stage and recycles these nutrients for the autotrophic/mixotrophic algal growth and vice versa. Additionally, algal culturing system 108 can also be designed to recycle the CO2 produced as a result of heterotrophic mode of algal growth to the autotrophic/mixotrophic growth, and can recycle the oxygen produced by the autotrophic/mixotrophic growth for heterotrophic growth. The recycling of nutrients for different trophic growth provides additional cost offsets made possible via algal culturing system 108.

As discussed in more detail below, the design of algal core 156 determines the amount of algae produced in AGC 152B based on the amount of CO₂ produced by AGC 152A or vice versa with oxygen production by AGC 152A fed to AGC 152B. For example, if AGC 152A produces about 1.8 tons of CO₂, one would expect that up to about 1 ton of dry algae biomass would be produced by AGC 152B.

AGC 152A has the advantage of accepting a myriad of inputs. For example, and as described previously, AGC 152A can use liquid manure waste as in input, or can use organic carbon from commercially available clean sources (e.g. sugars) or other waste streams, such as, but not limited to, grains spoilage from farms, brewery waste, liquids containing sugars from food waste, industrial wastes, or farm operation wastes, or a mixture of different wastes. Algal biomass production at AGC 152A can be maximized by using the naturally occurring or genetically enhanced algae strains, monoculture or polyculture, and/or other microbial strains such as bacteria and/or fungi that is best suited for the feedstock (e.g. sugars available from market or from waste sources) available at the target location. In other words, certain algae do better with certain carbon inputs than others. In an exemplary embodiment, the algae, Chlorella vulgaris, has been successfully cultured in dairy manure effluents. In another embodiment, AGC 152A can use and produce non-algae strains, such as the fungal strain, Trichoderma reesei, for converting aforementioned throughput feedstock into byproducts.

In an exemplary embodiment, AGC 152A includes heterotrophic algae, which is known to produce dense algae growth and a relatively high amount of useful by-products. Heterotrophic algae can be grown in fermenter(s), or closed or open system(s), or a combination or a hybrid form of the aforementioned. Standalone growth of heterotrophic algae is scalable in large sized vessels (such as, but not limited to, fermenters), and under heterotrophic growth conditions, respiration rates equal or exceed the theoretical minimum cost of biomass synthesis and biomass synthesis can achieve nearly the maximal theoretical efficiency.

One of the outputs (in addition to generated algal biomass for lipid extraction) of AGC 152A is an off-gas, CO₂, which is generated as a result of algae respiration due to organic uptake of carbon. The CO₂ generated by AGC 152A is used as an input for AGC 152B.

AGC 152B is designed to accept the output (which are typically byproducts) of AGC 152A. As such, AGC 152B can be a photoautotrophic, a mixotrophic, or a combination of both photoautotrophic and mixotrophic production systems of algae fed by the CO₂ produced by AGC 152A. AGC 152B can take the form of open, closed, or hybrid systems of algae growth and therefore can be implemented by various methodologies, such as, but not limited to, a tank, a bag, a fermenter, a tubular vessel, a plate, and a raceway, of any shape, size, or volume.

In an exemplary embodiment, AGC 152B uses clean sources of additional nutrients or captures nutrients from waste or wastewater streams, for example, but not limited to, anaerobically or aerobically digested effluent from dairy farms, industrial operations such as breweries, food waste, municipal waste, etc. The CO₂ input stream from various industrial operations, such as flue gases, supplied to second algal growth component 312 may contain other nutrients that promote algae biomass growth. While AGC 152B has been previously described as one or more of a photoautotrophic, mixotrophic, and heterotrophic algal growth, it could also include the cultivation of any biomass that requires the addition of inorganic carbon (CO₂) and/or organic carbon and/or nutrients (such as nitrogen and phosphorus and other micro or macro nutrients) for its growth.

In order to size algal core 156 (and ultimately determine an estimate of the total expected biomass (TEB) production of the system), the amount of algal biomass producible from AGC 152A at the site is determined based on the amount and type of throughput feedstock available, e.g., the amount available from on-site sources, brought from off-site sources, or combination of the two, to grow the respective algae type used in AGC 152A. For example, if the feedstock is nitrogen rich, algal types that may be paired with this feedstock include Chlorella vulgaris, Chlamydomonas reinhardtii, and Scenedesmus abundans. Alternatively, if the feedstock is phosphate rich, the algal types that may be paired with this feedstock include the bacteria Acinetobacter calcoaceticus or Acinetobacter johnsonii. Based upon the expected algal biomass producible from AGC 152A, an amount of CO₂ available to AGC 152B from AGC 152A can be determined. The available CO₂ and the amount of feedstock available to AGC 152B is determinative of the amount of biomass producible of AGC 152B. The TEB can then be determined as the sum of the algal biomass produced at AGC 152A and the biomass produced at AGC 152B.

The amount of biomass producible by either growth component, i.e., AGC 152A and AGC 152B, will be heavily influenced by the specific algae chosen for each respective component, and in the case of AGC 152B, the type of algae chosen. For example, a mixotrophic algal growth system requires less CO₂ because it requires greater organic carbon uptake when compared to a phototrophic system. Knowing the type of algal system chosen for AGC 152B (and the specific algae) can be used to determine the size or volume required for AGC 152B when implemented in the form of, for example, a closed photobioreactor, an open tank, a raceway, or a pond system. For example, if an output of 1000 tons of Chlorella vulgaris grown in AGC 152B (e.g. a photobioreactor) we would need at least 1800 tons CO2. That means we'll have to setup the AGC 152B system of the volume that can grow enough amount of heterotrophic biomass that can produce 1800 tons of CO2 because it is established fact that the photoautotrophic algae requires about 1.8 tons of CO2 to produce 1 ton of algae. In case of mixotrophic algal production, the CO2 requirement could be about 10 times lower.

In another embodiment the size of algal core 156 can be deduced inversely, e.g., first the maximum amount of biomass producible via AGC 152B on the site is determined (usually space/volume limited) based upon the type of algal system, inputs, and space/footprint available, then the CO₂ the requirements of the AGC 152B are determined, which can then be used to determine the composition and size of AGC 152A.

In yet another exemplary embodiment of algal core 156, an oxygen rich air supply from AGC 152A (when implemented as a photobioreactor as a result of photosynthesis by photoautotrophic or mixotrophic algae) is fed into AGC 152B (when implemented as a heterotrophic reactor to support growth of heterotrophic algae). This arrangement solves a major well-known constraint in closed photobioreactor systems caused by excessive oxygen production which has an adverse effect on the algae growth inside the photobioreactor.

In a further embodiment, an AGC 152A feeds AGC 152B while AGC 152B feeds AGC152. For example, AGC 152A may feed CO₂ to AGC152B, while AGC 152B, concomitantly, feeds O₂ to AGC 152B. Additional CO2 or O2 can be fed to the respective components for additional biomass production and carbon capture as desired.

Of the many advantages offered by SAS 100 and specifically by algal core 156, is the scalable nature of the system. Scalability is enhanced because heterotrophic algae (i.e., AGC 152A) is capable of dense growth when compared to photoautotrophic algae and certain mixotrophic algae. While density allows for greater biomass production per volume, heterotrophic algal growth in AGC 152A produces an off-gas, CO₂, and effluent containing nitrogen, phosphorus, and other components requiring treatment before discharge. However, the need and concomitant expense of treatment can be mitigated (or even eliminated in certain embodiments) by incorporating AGC 152B because the second algal growth component uses the CO₂ and effluent created by the AGC 152A, thus significantly reducing waste treatment costs while producing additional algal biomass.

While algal core 156 has been described above as a part of a larger system, e.g., SAS 100, algal culturing system 108, etc., it can also be implemented as a standalone system.

As shown in FIG. 3, an algal core 200 can also use post algal harvest liquid effluent obtained from AGC 204A as an input for AGC 204B so as to provide an additional supply of nutrients.

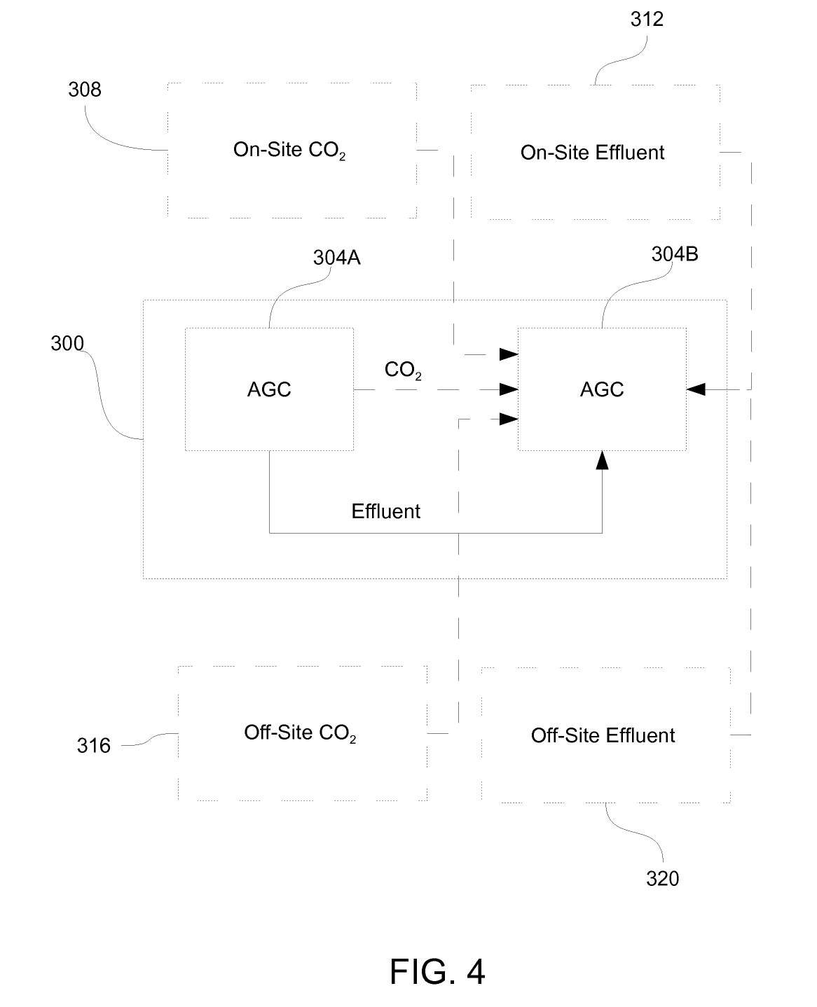

In yet another embodiment of algal core 200, and as shown in FIG. 4, a first AGC provides nutrients, but little if any (optionally) CO₂ to a second AGC. This embodiment may be useful at sites where other means of CO₂ capture, e.g., fossil fuel emissions capture, are available. Advantageously, using an algal core of this embodiment may also assist a CO₂ emitting facility keep CO₂ emissions within emission limits as the excess CO₂ can be fed to one of the AGC's.

Another embodiment of algal core, algal core 300, is shown in FIG. 4. In this embodiment, algal core 300 includes a pair of AGC's, AGC 304A and 304B. AGC 304B is optionally fed with various sources of CO₂ sources from either onsite resource 308, off-site resource 312, or from AGC 304A, or combinations of two or more of these CO₂ sources. For example, at a dairy farm, the anaerobically digested effluent containing nitrogen and phosphorus is on-site resource. The supplementary nutrient source from off-site could be the effluent from a creamery, cheese factory etc. FIG. 4 also shows AGC 304B being fed with additional sources of nutrients from either onsite resource 316 or off-site resource 320, including, but not limited to industrial waste, brewery waste and/or surplus, food waste and/or surplus, farm waste and/or surplus, and/or municipal waste.

Returning now to a discussion of FIG. 1, algal harvesting system 112 is used to collect the algal biomass generated by algal culturing system 108. In an exemplary embodiment of algal harvesting system 112 includes one or more solid separators 160, e.g., solid separator 160A and 160B, and a nutrient tank 164. Whether or not the output of AGC 152A or 152B should be sent to a separator 160 is determined by the type of output produced by the AGC. In an exemplary embodiment, and as shown in FIG. 1, AGC 152A produces a relatively low concentration algal biomass and thus separator 160A is used to concentrate the output of the AGC. In contrast, in an exemplary embodiment, AGC152B produces a relatively concentrated algal biomass output that can be sent directly to a biomass processor 168 (described in more detail below).

When algal harvesting system 112 is in use, algae biomass from AGC 152A is provided to solid separator 160A, which in this embodiment is a settling tank that allows the algae mass to settle to the bottom of the tank. In this embodiment, the bottom quarter of the settling tank (or so) is then physically separated from the rest of the settling tank's contents. The top ¾ of the settling tank (generally a liquid layer) is pumped out of solid separator 160A (and can be re-fed into either AGC 152A or AGC 152B, or sent to algal biomass processing system 116, as discussed below) leaving only the bottom algae concentrate which can be subsequently removed.

Algal solids (also referred to as concentrate) separated out by algae harvesting system 112 are sent to algal biomass processing system 116, which can be a standalone unit or a combination of Centrifugation, Filtration, Drying, Gravity settling, Microbial or Chemical based biomass aggregation, Flocculation and Sedimentation etc., to concentrate the algal solids. As shown in FIG. 1, algal biomass processing system 116 includes a pair of biomass processors 168 (biomass processors 168A and 168B). In an exemplary embodiment, biomass process is implemented as a separation funnel tank equipped with electrodes. In this embodiment, the algae concentrate from algal harvesting system 112 is gravity fed into the separation funnel tank. A current is then run through the algal concentrate, via the electrodes, causing individual algae cells to burst thereby releasing the lipids inside. The mixture within the separation funnel tank can then be allowed to separate into three layers, a solid layer (also referred to as “cake” layer), a water layer, and a lipid layer. The separation funnel tank can then be used to individually remove each layer for further processing or use. In another exemplary embodiment, biomass processing system 116 harvests algae from man-made water collection structure such as tanks, pits, ponds etc., or natural water bodies such as ponds, tributaries, lakes etc. in addition to being a part of SAS 100. The harvested algae can be become part of the algae cake and/or processed for different byproducts production such as fertilizer. In exemplary embodiments, biomass processing unit 116 is implemented as a centrifuge, or as a unit that is immersed or floats on water to harvest biomass. For instance, a biomass processing system 116 can be installed at a farm that has nutrient runoff collection pits installed, which captures farm runoff and thereby naturally produce additional algae and microbes. A biomass processing unit 116 can harvest these algae and microbes and add them to the algae cake. Algae cake with or without the addition of wild or naturally occurring algae can be dried or mixed with additional biomass for conversion into biofuel. In some of the instances of biomass processing unit 116 the algae cake is densified by the addition of a secondary material or a mix of materials such as sawdust, hay, grasses, pelletization or pucks waste or surplus, lumber waste or surplus, wood waste, or surplus etc. These densification processes may be beneficial to the renewable diesel production processes described below, to the formation of a storable form of fertilizer, or for the creation of combustible algal pellets for burning in gasifiers for heat. In some instances, algae cake alone or mixed with one or several materials, as described above, is pelletized or prepared into pucks, briquettes, pellets, etc., thereby providing increased storability. In another embodiment, algae cake is mixed with grasses grown on wasteland, or in buffer zones for capturing nutrients, e.g., miscanthus, switchgrass, etc., and then is formed into pellets, briquettes, pucks, etc.

Byproducts system 120 further treats the outputs received from algal biomass processing system 116. In an exemplary embodiment, from the lipid layer, crude algae oil is extracted with a solvent and a catalyst through a suitable process (chemical or non-chemical) at biofuel processor 172 so as to produce biodiesel and glycerol. In another exemplary embodiment, algae cake is converted into different forms of marketable fertilizer (either or both liquid and solid types). The solid fertilizer can be made into different forms such as powder, granular, pelleted, etc. and can include different proportions of nitrogen, phosphorous, and potassium (commonly combined and referred to as N-P-K). Producing algae fertilizer with marketable N-P-K concentrations has proved elusive. However, in certain embodiments of SAS 100, different algae types (monocultures, polycultures or aggregations of naturally occurring algae with or without other microbes or components), capable of capturing different fertilizer constituents (e.g., N, P, K), are grown separately either in the looped reactor or in combined or standalone autotrophic, mixotrophic or heterotrophic reactors or open ponds. Harvested algae can then be mixed in different proportions to obtain the marketable equivalent compositions of N-P-K, for example as in, Alfalfa meal (N-P-K: 2-1-2); Soymeal (7-2-1); and chicken manure (1.1-0.8-0.5). Algae fertilizer can also be enhanced by blends of different commercially or locally available materials for example, by adding trace minerals for creating algae-based seed starting mixes, or by adding potassium for creating certain desirable N-P-K composition. Granular fertilizer can be made using fertilizer processor 176, which, in an exemplary embodiment is a commercially available granulating machine. In an exemplary embodiment, algal cake with sufficient moisture is dried prior to granulation. It has been reported that solid form of fertilizer applications improve crop growth by providing the captured nutrients in a relatively stable and storable form, which is not possible with application of liquid manure on the land via manure spreader. This inefficiency exists, because there are only few time windows available for liquid manure spreading during the crop growth. However, using a storable, granulated form of algal-based fertilizer provides flexibility of application during the times when manure spreader cannot be used, such as for dressing the corn plants at the appropriate stage of their development. An environmental benefit, among others, of removal of nutrients via algal fertilizer is the reduction of nutrients runoff into natural water bodies. Moreover, cost offsets would be economically beneficial as fertilizer production produces an income stream for the farms or other businesses.

In yet another embodiment of byproducts system 120, biofuel processor 172 can convert algal biomass directly from algal culturing system 108, or through algal harvesting system 112, or algal biomass processing system 116 into ‘renewable diesel’ and byproducts via hydrogenation (treatment with addition of hydrogen) via processes such as, but not limited to, a) hydrothermal processing (for instance, by reacting the biomass on the order of 15 to 30 minutes in water at a very high temperature, typically 570° to 660° F. and pressure 100 to 170 atm standard atmosphere, enough to keep the water in a liquid state to form oils and residual solids); b) indirect liquefaction (for instance, a two-step process to produce ultra-low sulfur diesel by first converting the biomass to a syngas, a gaseous mixture rich in hydrogen and carbon monoxide, followed by catalytically conversion to liquids, the production of liquids is accomplished using Fischer-Tropsch (FT) synthesis as applied to coal, natural gas, and heavy oils; c) integrated catalytic thermochemical process such as integrated hydropyrolysis and hydroconversion (IH2); d) hydroprocessing (the hydrothermal liquefaction (HTL) of biomass provides a direct pathway for liquid biocrude production via two types of methods possible for conversion of fatty acids to renewable diesel: “high-pressure liquefaction” or “atmospheric pressure fast pyrolysis”).

Potable fresh water is produced as a byproduct of algal harvesting system 116 that can be recycled for other uses.

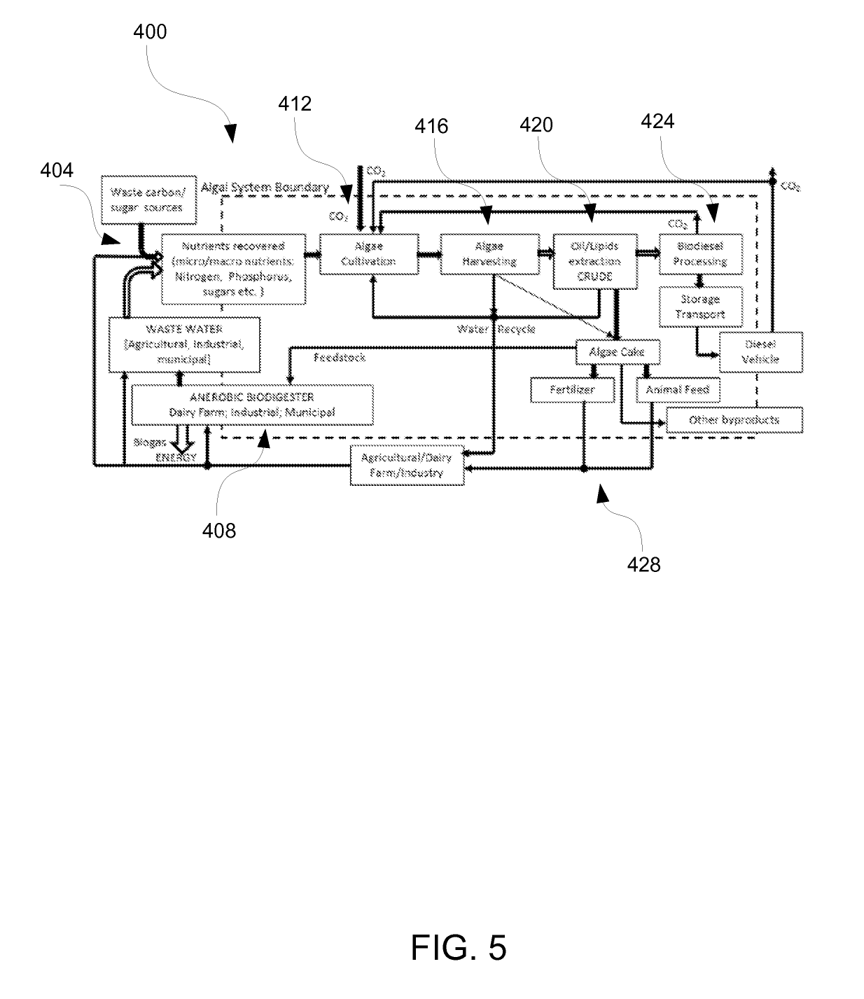

Turning now to FIG. 5, there is shown another exemplary symbiotic algae system, SAS 400, according to an embodiment of the present disclosure. At a high level, SAS 400 includes, but is not limited to, acquiring of feedstock inputs 404 from, for example, stakeholders, pretreater 408, algae cultivator 412, biomass harvester 416, oil extractor 420, byproducts manufacturer 424, and recycling of materials 428. Feedstock inputs 404 may be from a variety of stakeholders external to the SAS 400 operators, such as dairy manure waste generated at a farm (or in case of an industrial process, such as brewery, its generated wastes). Feedstock inputs 404 are processed through pretreater 408, which can be an anaerobic digester that in addition to generating effluent useful for algae cultivation, and also generate biogas and/or bio-electricity as alternative energy. Pretreater 408 is capable of generating an effluent/wastewater stream 432 with reduced odor and biochemical oxygen demand (BOD), which is advantageous for water quality. However, typically the pretreatment process of pretreater 408 does not remove nitrogen and phosphorus, which is a significant environmental issue, and due to government regulations typically requires further treatment for its safe discharge into natural water bodies. SAS 400 recovers the nutrients from effluent/wastewater via algae cultivator 412, which can be, for example, an embodiment of algal growth system 108 as described herein. At a desired time, the algal biomass produced by algae cultivator 412 is harvested at biomass harvester 416, which outputs lipids, water, and solids—each of which can be a useful produce or recycled within SAS 400. For example, lipids are extracted at oil extractor 420; water can be recycled into one or more of the other processes within SAS 400 such as algae cultivator 412 or back to one of the stakeholders (such as a dairy farm); and solids can be converted into animal feeds or fertilizers. The post-harvest algal biomass (also referred to as algae cake), and/or other algal biomass is utilized for production of additional useful by-products, such as fertilizer or animal feed depending on the throughput feedstock used. For example, algae biomass grown with dairy manure waste would be more appropriate as a fertilizer instead of animal feed due to required FDA compliance. In contrast, brewery effluent, which is a cleaner byproduct of beer processing and typically being food grade, can be used for producing algal biomass for high value animal feed. The crude oil extracted by oil extractor 420 goes through further processing to obtain desirable end products (biodiesel, oil-heat, jet fuel), and is then stored, transported and used in vehicles, planes or for heating purposes. Notably, as algae is a CO₂ sink, one can expect that at least a portion of the CO₂ generated by the local use of the aforementioned products can be recaptured by the algal biomass production process along with the CO₂ from the farm operations. Heat captured by pretreater 408 or from other onsite operations can be used as a heat input for algae cultivator 412, biomass harvester 416, oil extractor 420, and/or for sterilizing the algae cake that is used for animal feed production.

### Example 1

In this example, an algal core included a first algal growth component that was a heterotrophic component that included a heterotrophic algal strain and which generated and fed carbon dioxide to a second algal growth component was a photoautotrophic counterpart that included a photoautotrophic algal strain. It should be noted that the latter could be a photoautotrophic open pond/tank, or a hybrid system supporting photoautotrophic or mixotrophic growth.

Two sets of bioreactors were setup to represent a test (an embodiment of the algal core discussed above) and a control. The control system was a closed photobioreactor fed with ambient air. The test algal core included two closed reactors, a heterotrophic reactor and a photoautotrophic reactor (supporting heterotrophic and photoautotrophic algal growth, respectively), where the photobioreactor was connected to ambient air supply plus the additional carbon dioxide generated from the heterotrophic reactor produced as a result of fermentation process. Both control and test systems were run in duplicate under the same temperature conditions, utilized artificially prepared media, and algae inoculums (also referred to as algae starter). In this experiment, when compared to the photoautotrophic counterpart, only half of the amount of algae starter was used in the heterotrophic reactor so as to maintain control over the heterotrophic reactor process.

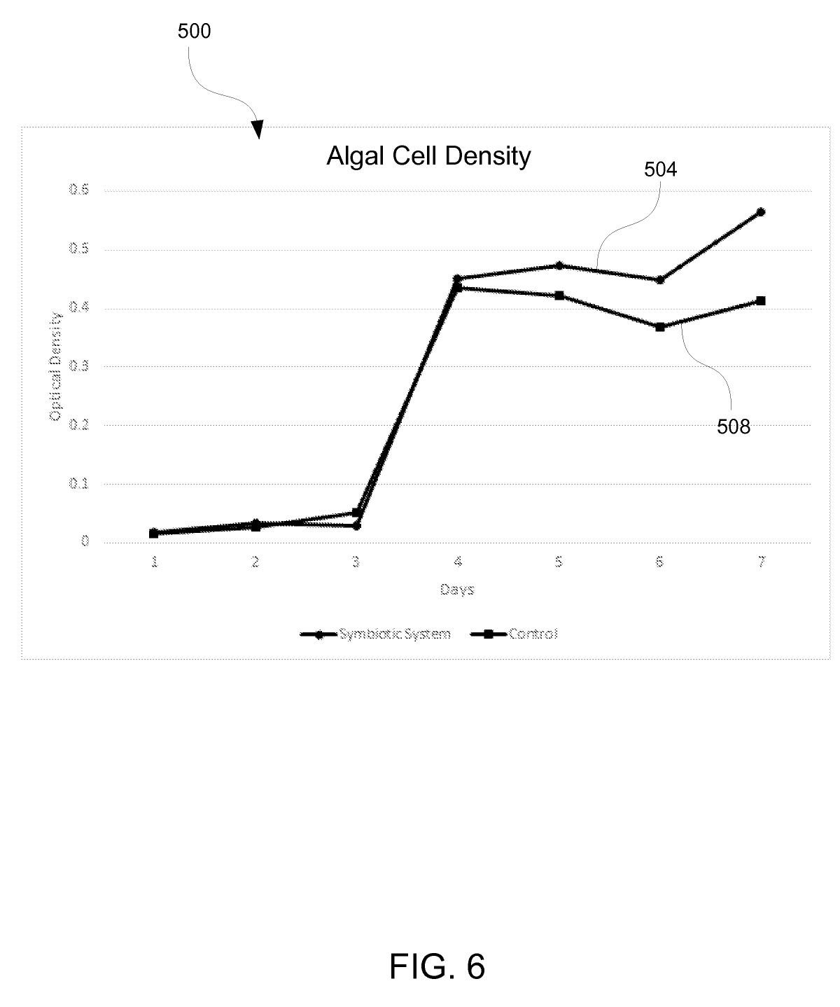

For the heterotrophic reactor, additional glucose was added to the artificial media, and the reactor was run without exposure to light. The photobioreactors had the same, constant light supply in both the test and the control batches. All reactors were regularly monitored for optical density, which indicates algal density (process discussed and shown in FIG. 6). Algal lipid content was monitored at the end of the log phase (day 4) and thereafter via confocal scanning laser microscope—a state-of-the-art multi-spectral imaging system using lipophilic dye. It was observed that the lipid content in the algal cells was negligible on day 4 and was highest on day 7 making it reasonable to harvest biomass on day 7. It should be noted that algae density can be strain and inoculum specific as some algae cultures may surpass the log phase earlier than 4 days, thereby making the harvest possible earlier than as shown in this example.

FIG. 6 shows a chart 500 of algae density (as measured by optical density) over time in days. Line 504 represents the test reactors and line 508 represents the control. As shown, very little algal density exists prior to day 4. After day 4, however, optical density substantially increases for both systems; however, algal density of the test system outpaces the control.

On the harvest day (day 7), the algal growth in the test algal core was found to be about 1.37 times higher (i.e., 37% more) than in the control reactor, which is considerable when extrapolated. For example, a typical harvested photoautotrophic algae on dry weight basis is in the range of 300 mg/L (0.3 gm/L) to a 1 gm or more in photobioreactors. Using the more conservative harvest estimate, i.e., the 0.3 gm/L scenario, and extrapolating to an exemplary and typical 2000 ton/day algal growth system, a conventional photobioreactor system (or open pond system) would produce about 728,000 tons of algae biomass for oil extraction annually, whereas the photoautotrophic algal biomass harvest in the algal core, as discussed above, would be about 994,728 tons-a 266,728 ton surplus harvest.

As noted above, the heterotrophic reactor received 50% of the algae starter compared to the photobioreactor; however, if both reactors included an equal amount of algal inoculum the amount of surplus algae from the heterotrophic reactor would be expected to double due to additional carbon dioxide generated by the heterotrophic reactor. If double the amount of heterotrophic algae was grown in the symbiotic system, this would contribute a surplus harvest of 3-4 times greater from the photobioreactor, thereby making the making the final surplus outcome about two or three times the harvest (i.e., about 74% to 111% more than the control). This example also illustrates how the volumes of the heterotrophic and photoautotrophic components in the symbiotic system could be customized to the algal harvest required from the two respective components. The surplus algal biomass generated could vary (lower or higher) in some embodiments depending on other factors such as media composition, light exposure, algae strain etc.

The examples and embodiments presented above could be applied to a variety of seed trains, where one system feeds a scaled up version of the system. Various combinations of an SAS, such as SAS 100, could be made with the other existing algal growth systems and/or microbial growth systems.

### Looped Algae Reactor Design Pattern (LARDP)

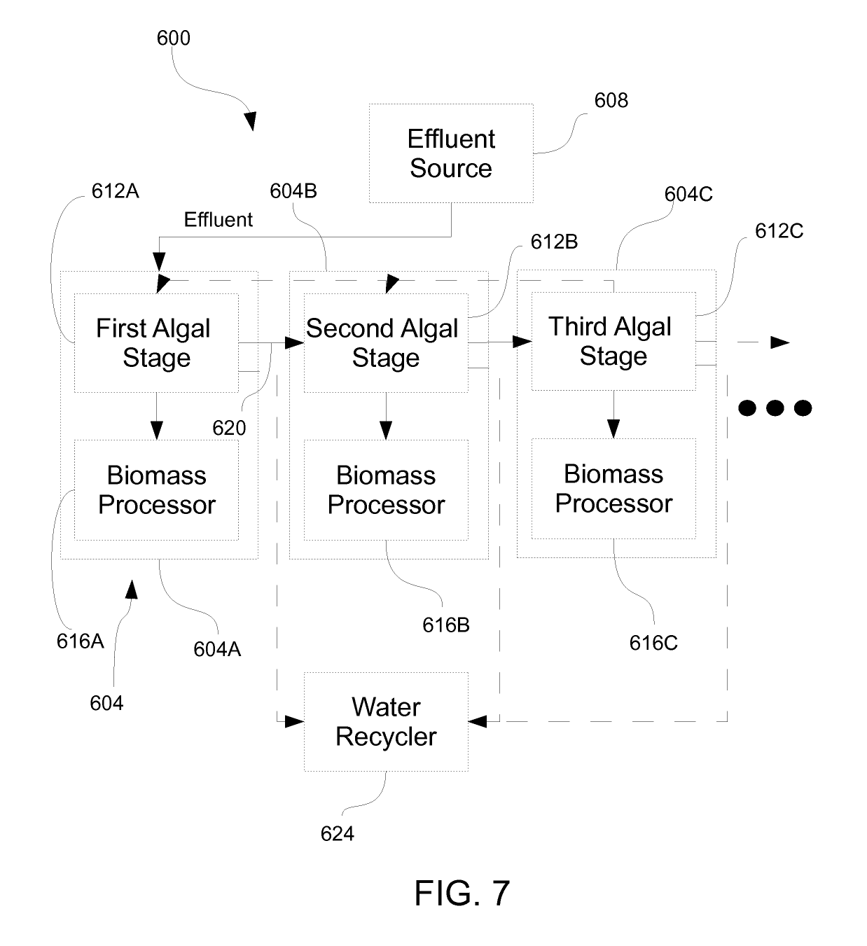

SAS 100 can, in certain embodiments, include a Looped Algae Reactor Design Pattern (LARDP) 600, as shown in FIG. 7. LARDP 600 is a process and/or a system that can be added/attached to algal cultivating system 108, algal cores 156, 200, or 300, or can be a standalone system attached to a waste treatment, wastewater treatment, remediation system for cleaning wastewater/effluent streams using one or more strains of microalgae (or other microbial organisms such as bacteria, fungi etc.), or to any algae-based or microbial-based process producing a target product or byproducts. At a high level, LARDP 600 uses a process of repeated cultivation of algae for co-product development and/or removal of nutrients for improving water quality of the effluent stream by growing algae biomass with or without other microorganisms.

LARDP 600 can include a series of nutrient extraction systems (NES) 604, such as first NES 604A and second NES 604B. Each NES 604 is designed to extract a certain type or types of components from an incoming effluent source 608, such as an algal effluent stream from an algal growth component, such as AGC 152A or 152B, or from other sources described herein. In an exemplary embodiment, first NES 604A includes a first algal stage 612 that receives, as an effluent stream as an input. First algal stage 612 is sized and configured to use microorganisms, such as those previously described herein, to extract from effluent stream 608 a certain type or types of components, such as, but not limited to, a nitrogen, a phosphorus, a heavy metal, a toxic component, a particular element (e.g. Ca, K, Mg, Na, Al, Fe, Mn, B, Cu, Zn, S, Pb, Cd, As), a complex element such as an antioxidant (e.g. astaxanthin), and a nuclear component. First algal stage 612 allows for the growth of the microorganisms and, in certain embodiments, can be similar in design to AGC 152B. At a desired time, the algal biomass produced by first algal stage 612 is harvested at biomass processor 616, which can be performed as described above. First algal stage 612 also produces an effluent 620, which is at least partially devoid of the component that first algal stage 612 was designed to remove. This effluent can proceed to one or more primary pathways. The effluent can 1) be recirculated back to first algal stage 612A for further extraction of components (not shown), 2) proceed to a water recycling unit 624 for further water treatment, 3) proceed to second algal stage 604B, and/or 6) return to algal cultivating system 108 (FIG. 1) (when LARDP 600 is coupled to such a system). In general, the concentration of the dominating component in the effluent 620 determines its destination. For example, if first algal stage 612 contained predominantly the alga Chlorella vulgaris which removed certain amount of nitrogen and phosphorus such that effluent 620 contains almost no nitrogen but still contained phosphorus, the effluent would likely travel to the 612 system containing the microorganisms capable of utilizing phosphorus more efficiently than Chlorella. vulgaris, such as, but not limited to Oscillatoria sp.

Second NES 604B and third NES 604C can be sized and configured to remove the same or a different type of component than that removed form first NES 604A. Second NES 604B thus can similarly include, a second algal stage 612B and a biomass processor 616B, and similarly third NES 604C can include, a third algal stage 612C and a biomass processor 616C. Additional stages 604 can be included to further extract components from effluent streams and recirculation to each stage in place in LARDP 600 can be performed. For example, if at first NES 604A, a first heavy metal is removed such that after entering the first NES it is present in the effluent stream in a lower concentration, the effluent can proceed to second NES 604B where another component, for example, a second heavy metal is removed to a lower concentration. The effluent from second NES 604B can then be recirculated to the first NES 604A for further removal of the first heavy metal, which is facilitated by the lower concentration of the second heavy metal.

In another exemplary embodiment, LARDP 600 is sized and configured to produce organic fertilizer from effluent steam 608. In this embodiment, at each NES 604 a desired fertilizer component is removed, e.g., nitrogen, phosphorous, potassium, etc. As each NES 604 allows for the harvesting of a concentrated amount of the desired component that is entrained within the organism, e.g., algae, in the NES, specific and fairly pure amounts of the component can be harvested and then mixed together to obtain the desired fertilizer product.

In use, when attached to an algal system, such as algal culturing system 108, microalgae disposed within LARDP 600 is cultivated in the effluent generated by the algae growth system. In this embodiment LARDP 600 is designed to remove undesirable substances such as, but not limited to, unwanted nutrients (e.g., nitrogen and phosphorus) and heavy metals. The biomass resulting from LARDP 600 can then be harvested from the waste water and, depending on what LARDP has been designed to extract, processed to produce useful products such as, but not limited to, fertilizer and compost, or can be used as feedstock for digesters producing energy such as biogas or bio-electricity. After removal of the undesirable substances as described above, the remaining wastewater can then further treated by cultivating the same or a similar strain of microalgae as used in algal growth system 108 for producing the primary product, or the remaining waster can be further treated by one or more different algae strain(s) used as a monoculture or a polyculture with or without other microorganisms such as bacteria or fungi to further remove nutrients (e.g., nitrogen and phosphorus) or heavy metals or any other undesirable components present in the wastewater generated. LARDP 600 can be repeated in one or more stages with same or different strains of algae and/or bacteria and/or fungi or any other organisms compatible with algal strains, grown as a monoculture or poly culture in any type of algal growth system until the desired level of water quality is reached.

The number of NES 604s used in LARDP is determined by the number of desired removable elements in the effluent(s) that require capturing using microalgae or microbes and the desired water quality.

In an embodiment of the system, the one or more 604 stages in LARDP can be optionally combined or replaced by other processes such as multiple screening systems, decanting centrifugation, polymer flocculation, ammonia stripping, struvite formation, nitrification/de-nitrification etc. Modifications of these processes can also be used for enhancing the whole process of nutrient removal.

LARDP 600 can be useful in the creation of products, including, but not limited, to biofuels, fertilizer, animal feed, and cosmetics. The organisms cultivated in LARDP 600 can be cultivated under a green house or other similarly enclosed environment, so as to prevent contamination by competitive microorganisms while admitting light. LARDP 600 can be implemented in, for example, vertical freestanding tanks, raceway style ponds, or tracks.

Additional useful byproducts from SAS 100, include the production of clean carbon dioxide (as compared to the CO₂ captured from flue gases) generated from an algal growth component, such as AGC 152A, which, while discussed previously as supporting AGC 152B, can also be captured and used for other applications needed a clean source of CO₂, e.g., medical applications, electronics, laboratories, etc. Alternatively, the CO₂ can be used for algal inoculum-preparation (a highly concentrated algae culture typically used for seeding a larger scale system) especially to generate light-dependent inoculum for seeding a system or sub-system.

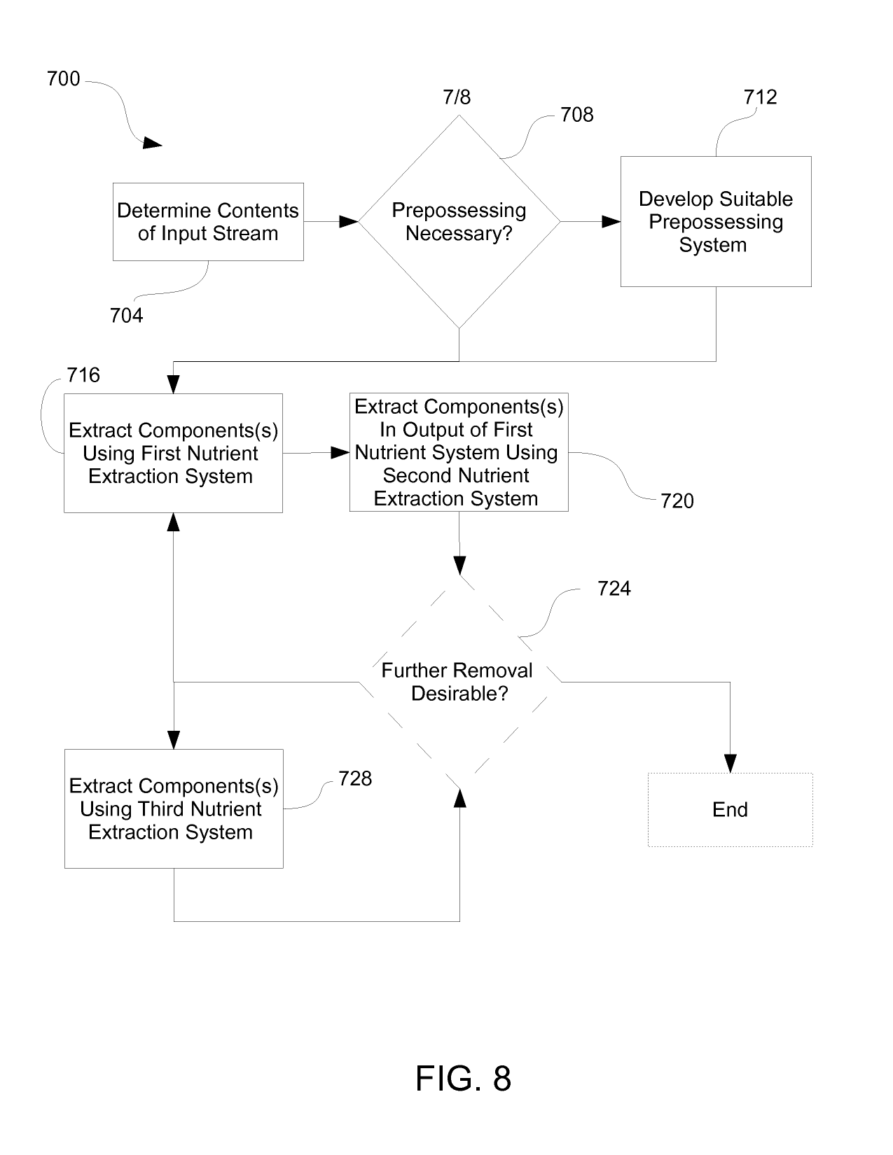

FIG. 8 shows a process 700 for removing contaminants from a waste or effluent stream. At step 704, the content of the waste or effluent stream is determined. While typical nutrients, such as nitrogen and phosphorous are likely to be found, the stream may also include heavy metals or other nutrients that a desirably removed from the stream before the stream is put to further use or otherwise treated. Determining which nutrients and other particles are a part of the waste or effluent stream will assist in determining the type of nutrient extraction system, such as one of the NES 504s discussed above, to implement.

At step 708 it is determined whether any preprocessing is necessary prior to the stream entering the first NES. Preprocessing may be necessary if the stream contains significant solids or too much liquid. If preprocessing is necessary, process 700 proceeds to step 712 where a suitable preprocessing system is developed. Exemplary preprocessing systems are solids treatment unit 140 and separator 136 as discussed above with reference to FIG. 1. If no preprocessing is necessary, process 700 proceeds to step 716.

At step 716, a first NES is used to extract components from the waste or effluent stream. In an exemplary embodiment, first NES is sized and configured to focus on a relatively small number of components for extraction. For example, if the input waste or effluent stream is nitrogen rich, first NES may be configured to include an algal component that is primarily effective at removing a substantial portion of the nitrogen from the waste or effluent stream. The output of first NES is then provided to a second NES at step 720 for extraction of another component of the original waste or effluent stream.

At optional step 724, a determination is made as to whether further removal of nutrients from the output of step 720 is desired. As part of step 724 a determination of the composition of the output of step 720 may be completed and may be used when the effectiveness of steps 716 and 720 and may be necessary so as to determine where, if anywhere, the output of step 720 should be sent. For example, in order to effectively remove a heavy metal from a waste stream, it is generally beneficial to remove nutrients that are in the stream in significant amounts. Therefore, if, for example, the output of step 720 included significant amounts of a nutrient, e.g., nitrogen, that would render extraction of the heavy metal difficult or inefficient, step 724 would determine that the stream should be sent to an NES that will efficiently remove more nitrogen (e.g., step 716). However, if removal of a different component is desired, process 700 may proceed to step 728 where a third NES is used to extract components form the output stream. If no further extractions are necessary, the process ends.

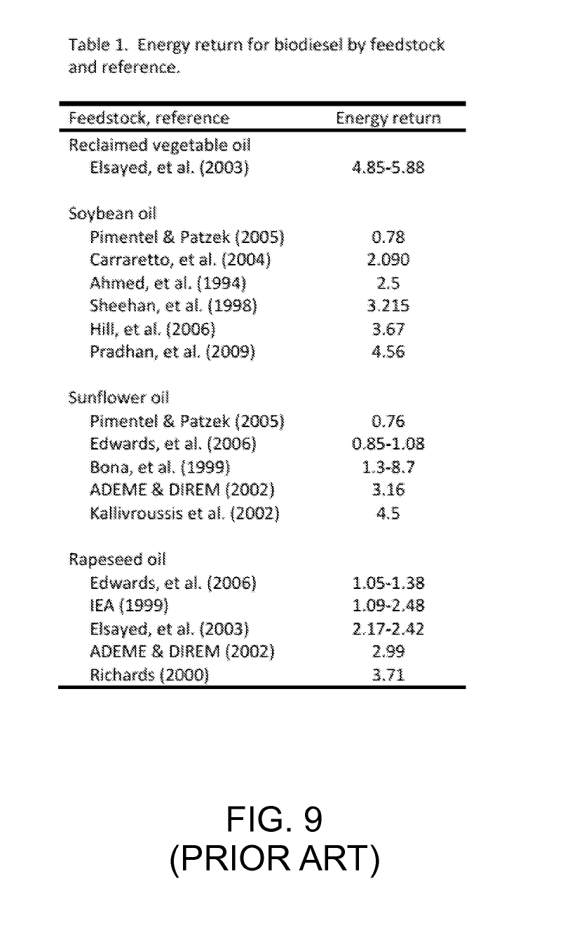

Turning now to a discussion of FIG. 9, there is shown energy return values (EROI) for biodiesel by feedstock. The EROI is calculated as the ratio between the energy produced and the energy consumed by a system, and is generally considered a critical measure for evaluating the net energetic profitability of that system. As the EROI increases, the energetic profitability of that energy system also increases. For any feedstock (e.g., algae, soybean oil, etc.) or combination of feedstocks (e.g., SAS 100) to be a net energy source, the EROI to operate the entire associated production system(s) must be greater than 1. However, historically, the EROI of viable energy sources has been much greater than 1 and, therefore, practical deployment of an energy source typically requires an EROI much greater than 1. For instance, the EROI has been used to characterize several conventional fuels; for example, for coal, oil and gas, and corn ethanol, the second-order EROI has been estimated to be ˜80 (at the mine), ˜15 (at the well), and ˜1 (at the biorefinery). Delivered gasoline (considering the entire supply chain) has reported an overall EROI of around 5 to 10.

As shown in FIG. 9, the EROI range is a low of 0.76 for sunflower oil to a high of about 5.88 for reclaimed vegetable oil. In comparison, certain embodiments of SAS 100 (varying pretreatment and algae types) obtained an energy return values of about 1, 11, and 40. Under certain conditions it can go even higher.

In an exemplary aspect, a symbiotic algae system is disclosed that comprises: a first algal growth component, wherein the first algal growth component includes a heterotrophic organism, and wherein the first algal growth component produces a first effluent and an off-gas; and a second algal growth component is fluidly coupled to the first algal growth component, and the second algal growth component including at least one organism from the group of: a photoautotrophic organism, a mixotrophic organism, and a heterotrophic organism, and wherein the second algal growth component receives, as an input, the first effluent and the off-gas and produces a second effluent. In the symbiotic algae system, the first algal growth component can receive, as a first input, an effluent input or a waste input. In the symbiotic algae system, the second algal growth component can receive, as a second input, an effluent input or a waste input. The symbiotic algae system can further include a waste nutrient preparation sub-system fluidly coupled to the first algal growth component. In the symbiotic algae system, the waste nutrient preparation sub-system can receive an effluent input, a fresh water input, and waste input, and outputs an effluent suitable for use by the first algal growth component. In the symbiotic algae system, the waste nutrient preparation sub-system is a manure settling and solid's preparation unit that outputs liquid manure waste to the first algal growth component. The symbiotic algae system can further include an algal harvesting system having at least one separator, wherein the algal harvesting system is fluidly coupled to the first algal growth component and/or the second algal growth component. The symbiotic algae system can have an EROI greater than 10. The symbiotic algae system can have an EROI of about 40. The symbiotic algae system can further comprise a third algal growth component, wherein the third algal growth component includes at least one organism from the group of: a photoautotrophic organism, a mixotrophic organism, and a heterotrophic organism, and wherein the third algal growth component receives, as an input, the second effluent. The symbiotic algae system can further comprise at least one biomass processing unit, the biomass processing unit sized and configured to extract lipids from at least one of the first algal growth component and the second algal growth component.

In another exemplary aspect, a symbiotic algae system is disclosed that comprises a first algal growth component, wherein the first algal growth component includes a heterotrophic organism, and wherein the first algal growth component produces an first effluent and an off-gas; and a second algal growth component fluidly coupled to the first algal growth component, wherein the second algal growth component includes at least one organism from the group of: a photoautotrophic organism, a mixotrophic organism, and a heterotrophic organism, and wherein the second algal growth component receives, as a first input, the first effluent and the first off-gas and produces an second effluent and a second off-gas; and wherein the second effluent and the second off-gas are received as inputs to the first algal growth component. In the symbiotic algae system, the first algal growth component can receive, as an additional input, an effluent input or a waste input, and wherein the additional input and the second effluent include a nitrogen and a phosphorous. In the symbiotic algae system, the first algal component can removes a portion of the nitrogen and the phosphorous from the second input and the additional input. The symbiotic algae system can further comprise a third algal growth component, wherein the third algal growth component includes at least one organism from the group of: a photoautotrophic organism, a mixotrophic organism, and a heterotrophic organism, and wherein the third algal growth component receives a portion of the second effluent. The symbiotic algae system can further comprise at least one biomass processing unit, the biomass processing unit sized and configured to extract lipid/oil from at least one of the first algal growth component and the second algal growth component. The symbiotic algae system can have an EROI greater than 10. The symbiotic algae system can have an EROI of about 40.

In yet another exemplary aspect, a symbiotic algae system can comprise: a waste nutrient preparation sub-system; an algal culturing system including: a first algal growth component fluidly coupled to said waste-nutrient preparation sub-system, wherein the first algal growth component includes a heterotrophic organism, and wherein the first algal growth component produces a first effluent and an off-gas; and a second algal growth component, wherein the second algal growth component includes at least one organism from the group of: a photoautotrophic organism, a mixotrophic organism, and a heterotrophic organism, and wherein the second algal growth component receives, as an input, the effluent and the off-gas and produces a second effluent; and an algal harvesting system fluidly coupled to said algal culturing system; an algal biomass processing system fluidly coupled to said algal harvesting system; and a byproducts system fluidly coupled to said algal biomass processing system. In the symbiotic algae system, the waste nutrient preparation sub-system can receive, as an input, an effluent input or a waste input.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A symbiotic algae system comprising:
a first algal growth component, wherein the first algal growth component includes a heterotrophic organism, and wherein the first algal growth component produces a first effluent and an off-gas; and
a second algal growth component is fluidly coupled to the first algal growth component, and the second algal growth component including at least one organism from the group of: a photoautotrophic organism, a mixotrophic organism, and a heterotrophic organism, and
wherein the second algal growth component receives, as an input, the first effluent and the off-gas and produces a second effluent.

2. A symbiotic algae system according to claim 1, wherein the first algal growth component, receives, as a first input, an effluent input or a waste input.

3. A symbiotic algae system according to claim 2, wherein the second algal growth component receives, as a second input, an effluent input or a waste input.

4. A symbiotic algae system according to claim 1, further including a waste nutrient preparation sub-system fluidly coupled to the first algal growth component.

5. A symbiotic algae system according to claim 4, wherein the waste nutrient preparation sub-system receives an effluent input, a fresh water input, and waste input, and outputs an effluent suitable for use by the first algal growth component.

6. A symbiotic algae system according to claim 4, wherein the waste nutrient preparation sub-system is a manure settling and solid's preparation unit that outputs liquid manure waste to the first algal growth component.

7. A symbiotic algae system according to claim 1, further including an algal harvesting system having at least one separator, wherein the algal harvesting system is fluidly coupled to the first algal growth component and/or the second algal growth component.

8. An algal cultivating system according to claim 1, wherein the system has an EROI greater than 10.

9. An algal cultivating system according to claim 1, wherein the system has an EROI of about 40.

10. A symbiotic algae system according to claim 1, further comprising a third algal growth component, wherein the third algal growth component includes at least one organism from the group of: a photoautotrophic organism, a mixotrophic organism, and a heterotrophic organism, and wherein the third algal growth component receives, as an input, the second effluent.

11. A symbiotic algae system according to claim 1, further comprising at least one biomass processing unit, the biomass processing unit sized and configured to extract lipids from at least one of the first algal growth component and the second algal growth component.

12. A symbiotic algae system comprising:
a first algal growth component, wherein the first algal growth component includes a heterotrophic organism, and wherein the first algal growth component produces an first effluent and an off-gas; and
a second algal growth component fluidly coupled to the first algal growth component, wherein the second algal growth component includes at least one organism from the group of: a photoautotrophic organism, a mixotrophic organism, and a heterotrophic organism, and
wherein the second algal growth component receives, as a first input, the first effluent and the first off-gas and produces an second effluent and a second off-gas; and
wherein the second effluent and the second off-gas are received as inputs to the first algal growth component.

13. A symbiotic algae system according to claim 12, wherein the first algal growth component, receives, as an additional input, an effluent input or a waste input, and wherein the additional input and the second effluent include a nitrogen and a phosphorous.

14. A symbiotic algae system according to claim 13, wherein the first algal component removes a portion of the nitrogen and the phosphorous from the second input and the additional input.

15. A symbiotic algae system according to claim 12, further comprising a third algal growth component, wherein the third algal growth component includes at least one organism from the group of: a photoautotrophic organism, a mixotrophic organism, and a heterotrophic organism, and wherein the third algal growth component receives a portion of the second effluent.

16. A symbiotic algae system according to claim 12, further comprising at least one biomass processing unit, the biomass processing unit sized and configured to extract lipids from at least one of the first algal growth component and the second algal growth component.

17. A symbiotic algae system according to claim 12, wherein the system has an EROI greater than 10.

18. A symbiotic algae system according to claim 12, wherein the system has an EROI of about 40.

19. A symbiotic algae system comprising:
a waste nutrient preparation sub-system;
an algal culturing system including:
a first algal growth component fluidly coupled to said waste-nutrient preparation sub-system, wherein the first algal growth component includes a heterotrophic organism, and wherein the first algal growth component produces a first effluent and an off-gas; and
a second algal growth component, wherein the second algal growth component includes at least one organism from the group of: a photoautotrophic organism, a mixotrophic organism, and a heterotrophic organism, and
wherein the second algal growth component receives, as an input, the effluent and the off-gas and produces a second effluent; and
an algal harvesting system fluidly coupled to said algal culturing system;
an algal biomass processing system fluidly coupled to said algal harvesting system; and
a byproducts system fluidly coupled to said algal biomass processing system.

20. A symbiotic algae system according to claim [0066], wherein the waste nutrient preparation sub-system receives as an input an effluent input or a waste input.
